(19) United States
(12) Patent Application Publication
(10) Pub. No.: US 2018/0184727 A1
(43) Pub. Date: Jul. 5, 2018

(54) SMART WEARABLE DEVICE

(71) Applicant: Flex Ltd., Singapore (SG)

(72) Inventors: Orna Petruschka, Kiryat Tivon (IL); Shimon Blitshtein, Nesher (IL); Yitzchak Shpitzer, Haifa (IL)

(21) Appl. No.: 15/840,964

(22) Filed: Dec. 13, 2017

(30) Foreign Application Priority Data
Dec. 29, 2016 (IL) 249867

(51) Int. Cl. A41D 1/00; G06F 1/16; H02J 7/02; H05K 1/02; H05K 1/18; F21S 4/10; A61B 5/01; A61B 5/00; G01K 13/00; G01L 5/00

(52) U.S. Cl. CPC A41D 1/002; G06F 1/163; H02J 7/025; H05K 1/028; H05K 1/18; H04W 4/008; A61B 5/01; A61B 5/6804; G01K 13/002; G01L 5/00; A61B 2562/0247; F21S 4/10

(57) ABSTRACT

A wearable device is provided having electronic components disposed therein. The electronic components maybe further attached to or disposed on a flexible circuit board and comprise inputs, outputs (e.g., LEDs, displays, etc.) and wireless communications. A user may operate the wearable device via an application executing on a secondary device in communication with the wearable device.

### CROSS REFERENCE TO RELATED APPLICATION

The present application claims the benefits of Israel Application Serial No. 249867, filed Dec. 29, 2016 entitled “SMART WEARABLE DEVICE”, which is incorporated herein by this reference in its entirety.

### FIELD OF THE INVENTION

The disclosure relates generally to smart devices and particularly to smart wearable devices.

### BACKGROUND

Until recently, clothing has been limited to serving functional and stylistic needs. However, despite the advances is clothing, textile, and computing technology, opportunities remain to address certain problems of the prior art and advance the state of the art.

### SUMMARY

These and other needs are addressed by the various embodiments and configurations of the present disclosure.

A smart wearable sensing and illumination integrated fabric component that can be embedded, placed, or designed into an apparel product, such as a sleeve of a jacket or shirt, a pant leg of shorts or pants, or in the body of a shirt, jacket, coat, or dress, is provided. The component can sense user biometrics and transmit the biometrics to a connected wireless computing device, such as a smart phone, tablet computer, and the like, and provide illumination for identification in darkness or low light conditions or illumination to indicate a sensing function is occurring.

The present disclosure can provide a number of advantages depending on the particular configuration. The component can not only sense user biometrics but also provide illumination to assist the user or wearer. The intelligent fabric component system can offer a miniaturization solution for integrated body wear and other applications where human interface for biometrics and safety is needed.

Apparel is becoming a based medium for wearable technology. As usable real estate areas on the body becomes limited for wearable technology sensing, miniaturization and smart integrated components will be designed into various apparel items such as shirts, jackets, pants, dresses, leggings, socks, etc.

In addition to sensing for personal biometrics as well as illumination for individual safety, miniaturization of smart sensing and illumination will expand into safety wear for first responders and other safety professionals, into automobiles as car seats, arm rests, or other human interface area where the health or monitoring of the users biometrics may be needed. Embodiments disclosed of an intelligent fabric component system offers a miniaturization solution for integrated body wear and other applications where human interface for biometrics and safety is needed.

These and other advantages will be apparent from this disclosure.

The phrases “at least one”, “one or more”, “or”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C”, “A, B, and/or C”, and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any computer-readable storage and/or transmission medium that participate in providing instructions to a processor for execution. Such a computer-readable medium can be tangible, non-transitory, and non-transient and take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

A “computer readable storage medium” may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may be any computer readable medium that is not a computer readable storage medium and that can communicate, propagate, or transport a program for use by or in connection with an instruction execution system, apparatus, or device. A computer readable signal medium may convey a propagated data signal with computer readable program code embodied therein, for example, in baseband or as part of a carrier wave. Such a propagated signal may take any of a variety of forms, including, but not limited to, electromagnetic, optical, or any suitable combination thereof. Program code embodied on a computer readable signal medium may be transmitted using any appropriate medium, including but not limited to wireless, wireline, optical fiber cable, RF, etc., or any suitable combination of the foregoing.

The terms “determine”, “calculate” and “compute,” and variations thereof, as used herein, are used interchangeably and include any type of methodology, process, mathematical operation or technique.

The term “means” as used herein shall be given its broadest possible interpretation in accordance with 35 U.S.C., Section(s) 112(f) and/or 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “module” as used herein refers to any known or later developed hardware, software, firmware, artificial intelligence, fuzzy logic, or combination of hardware and software that is capable of performing the functionality associated with that element.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein. By way of example, the phrase from about 2 to about 4 includes the whole number and/or integer ranges from about 2 to about 3, from about 3 to about 4 and each possible range based on real (e.g., irrational and/or rational) numbers, such as from about 2.1 to about 4.9, from about 2.1 to about 3.4, and so on.

The preceding is a simplified summary of the disclosure to provide an understanding of some aspects of the disclosure. This summary is neither an extensive nor exhaustive overview of the disclosure and its various embodiments. It is intended neither to identify key or critical elements of the disclosure nor to delineate the scope of the disclosure but to present selected concepts of the disclosure in a simplified form as an introduction to the more detailed description presented below. As will be appreciated, other embodiments of the disclosure are possible utilizing, alone or in combination, one or more of the features set forth above or described in detail below.

### BRIEF DESCRIPTION OF THE DRAWINGS

The present disclosure is described in conjunction with the appended figures:

FIG. 1 depicts a first side of a wearable device in accordance with embodiments of the present disclosure;

FIG. 2 depicts a second side of a wearable device in accordance with embodiments of the present disclosure;

FIG. 3 depicts a method to charge a wearable device in accordance with embodiments of the present disclosure;

FIG. 4 depicts a method to operate a wearable device in accordance with embodiments of the present disclosure;

FIG. 5 depicts wireless connectivity between the wearable device and a portable communications device in accordance with embodiments of the present disclosure;

FIG. 6 depicts wireless connectivity between wearable devices in accordance with embodiments of the present disclosure; and

FIG. 7 depicts connectivity between the wearable and various devices in accordance with embodiments of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

The exemplary systems and methods of this disclosure will also be described in relation to analysis software, modules, and associated analysis hardware. However, to avoid unnecessarily obscuring the present disclosure, the following description omits well-known structures, components, and devices that may be shown in block diagram form, and are well known or are otherwise summarized.

For purposes of explanation, numerous details are set forth in order to provide a thorough understanding of the present disclosure. It should be appreciated, however, that the present disclosure may be practiced in a variety of ways beyond the specific details set forth herein.

In one embodiment an assembly, integration and encapsulation of electronics in fabric is disclosed which includes one or more of: printed electronics circuitry to electrically connect subcomponents listed below; printed circuit board (rigid) and flexible printed circuits or rigid flexible printed Circuits to electrically connect subcomponents listed below;

lighting/illumination components (such as Light Emitting Diodes (LEDs), electroluminescent lighting (EL) or light pipes), LED matrix display; wireless charging using an integrated inductive charging subcomponent; magnetic charging component, Bluetooth Low Energy (BLE) or other wireless based connectivity; antenna(s);
heating component based on resistance based heating elements; pressure, temperature, moisture, heart rate, and other sensors form factor; on-off switch; battery (rechargeable) and/or energy harvesting system to power the integrated fabric based component system; and/or interface to tablet or smart phone or other product that has connectivity link with an app that allows control of the intelligent fabric component

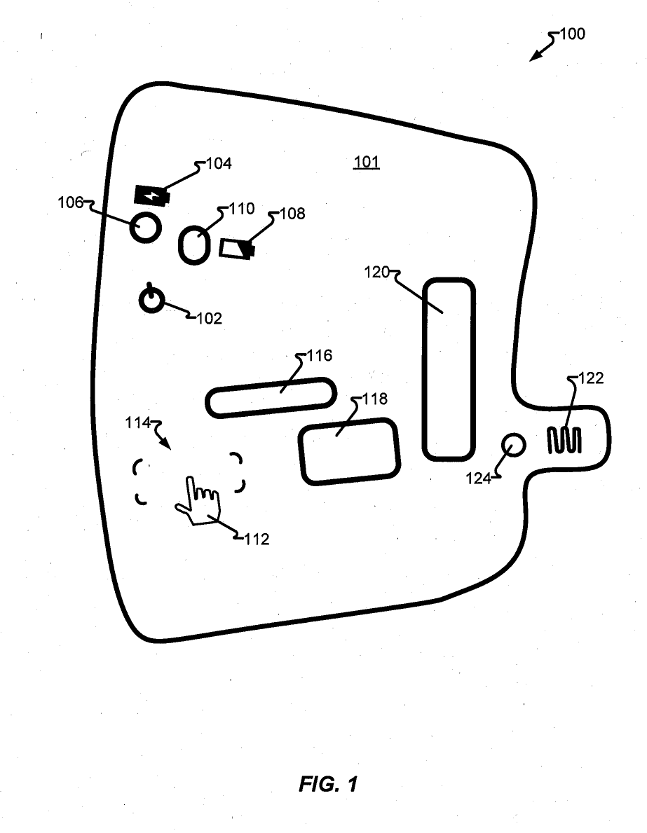

FIG. 1 depicts first side 101 of wearable device 100 in accordance with embodiments of the present disclosure. In one embodiment, wearable device 100 is a thin, flexible form factor, such as fabric or textile and is operable to attach to a user and/or attach to or be embedded into a user's clothing. Wearable device 100 comprises a flexible printed circuit board (PCB) assembly, illuminating components, pressure sensor, LED matrix display, rechargeable battery, wireless charging antenna, and Bluetooth Low Energy (BLE) connectivity.

Wearable device 100 comprises a number of components. The number of components may be interconnected with flexible wiring for the conduction of power and/or data signals between any two or more of the number of components. Alternatively, at least two of the number of components may utilize wireless connectivity for the exchange of data signals, such as radio frequency, infrared, sound, and/or other mechanical or electromagnetic medium. In one embodiment, components may include one or more of on-off button 102, charging indicator icon 104, charging indicator 106, battery status icon 108, battery status indicator 110, plastic fiber optic (POF) lighting 116, display 118, electroluminescent tape (EL) lighting 120, wakeup button 124, heater/temperature sensor 122, pressure sensor icon 112, and/or pressure sensor area 114.

In one embodiment, the shape of wearable device 100 may be suitable for use on a human's forearm.

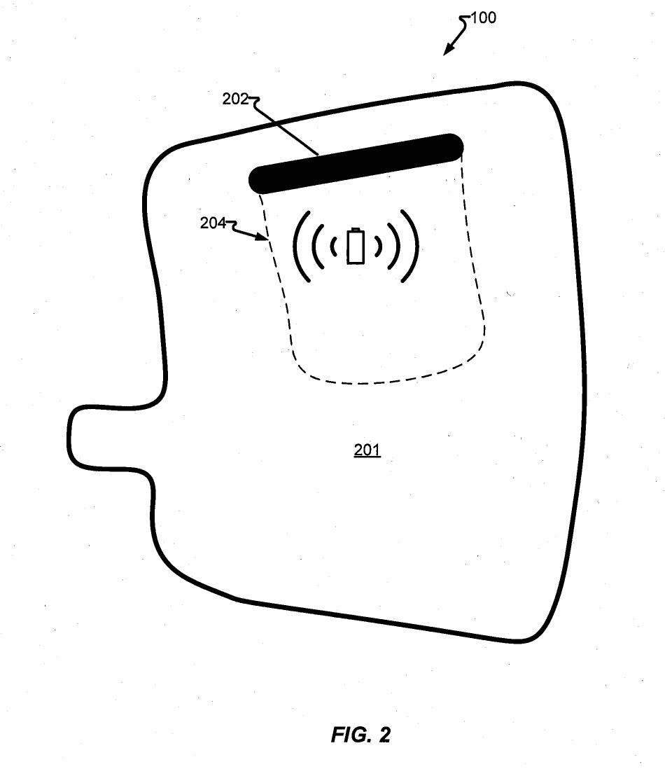

FIG. 2 depicts second side 201 of a wearable device 100 in accordance with embodiments of the present disclosure. In one embodiment, wearable device 100 may comprise a wireless recharging area icon 204 and pocket 202 to accommodate a wireless charger.

In one embodiment, pressure sensor area 114 comprises a pressure sensor located substantially at pressure sensor area 114, such as a PRI solution from Sensing Tex. While embodiments herein are preferably directed towards components disposed in the interior of wearable device 100, disposing one or more of the number of components on the exterior, such as on first side 101 or second side 201, are also contemplated by the embodiments provided herein. The pressure sensor disposed at pressure sensor area 114 may comprise a piezoresistive ink (PRI) which may be connected to the PCB by electrically conductive adhesive.

In another embodiment, POF lighting 116 may comprise optical fibers, such as organic based materials. The organic based materials may comprise polymethylmethacrylate (PMMA) or equivalent or inorganic based materials such as glass, such as Corning's Fibrance material and may further be illuminated by RGB LEDs, laser or other light source. EL lighting 120 may comprise a printed EL illumination component. Heater/temperature sensor 122 may comprise a thin flexible heating element, such as from PTI Pelonis Technology Inc.

In another embodiment, one or more of the number of components may be attached or connected to a printed circuit board assembly (PCBA). The PCBA may comprise a thin double layer FPC (flexible printed circuit), which may have a thickness of 76 μm. The PCBA may further comprise a MCU, which may further comprise an ultra-low power MCU by ST Microelectronics. In another embodiment, display 118 may comprise an LED matrix display. Wireless recharging area icon 204 may indicate the location of a wireless charger, such as provided by Texas Instruments. A BLE connectivity module may comprise a very low power module, such as BT Smart v4.1 by ST Microelectronics. EL lighting 120 may be signaled by an EL driver, such as high voltage, dimmable EL lamp driver, provided by “Microchip.”

One or more of the number of components of wearable device 100 may be powered by a power supply, such as a thin flexible battery, which may be the Prologium, ceramic lithium or imprint energy using zinc-based technology. Other sources of electrical power may be provided to the number of components directly, or via a battery or other storage component, such as solar, mechanical (e.g., piezoelectric) generators, chemical reactions, etc. A wireless charging antenna, such as to be co-located with recharging area icon 204, may be provided by a flexible, bendable form factor of copper, silver, or aluminum, such as may be provided by TDK.

In another embodiment, the number of components or at least a portion of one or more of the number of components, of wearable device 100 may be encapsulated between two plastic films. The film may include, but is not limited to, 3M 8673 Polyurethane Protective Tape and Argotec 46510 Thermoplastic Polyurethane. In another embodiment, high form components, such as those protruding out of the encapsulation, maybe coated with an epoxy, including but not limited to 3M DP105 epoxy adhesive, such as to maintain waterproofing. Ultrasonic welding and/or other fusing/adhering may be implemented such as to increase water proofing of the encapsulation of the one or more components.

In another embodiment, the encapsulated assembly of one or more of the number of components is integrated between two layers of fabric by using 3M 9627 adhesive and the fabric may then be decorated with embroidery or other indicia applied by a computerized equipment. Additionally or alternatively, a layer of foam or similar material may be utilized beneath the fabric to provide a softer feel and/or to conceal the electronic components.

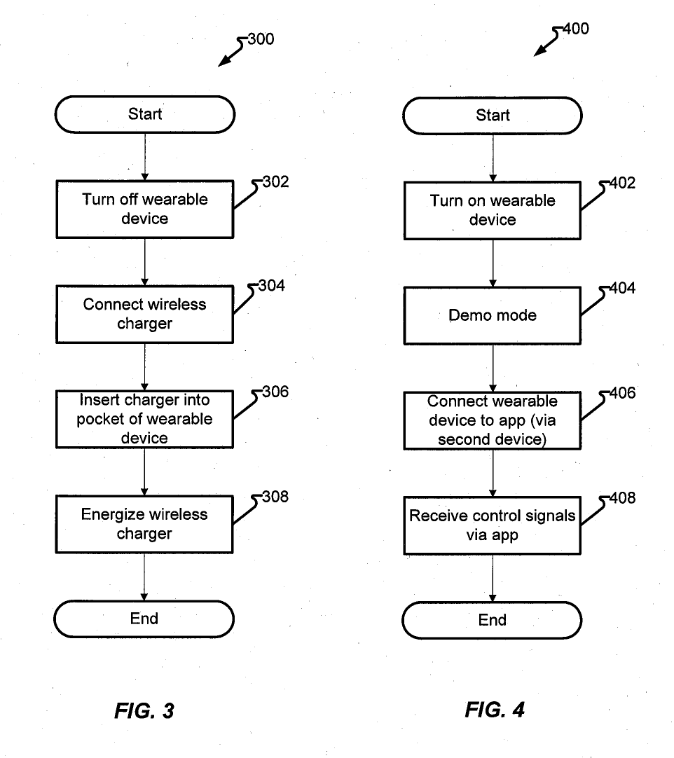

FIG. 3 depicts method 300 to charge wearable device 100 in accordance with embodiments of the present disclosure. In one embodiment, method 100 starts and step 302 turns off, such as via a user input to power on-off button 102, or timer, signal, etc., such as to cause wearable device 100 to be substantially deenergized. Optionally, step 302 may confirm wearable device is powered off or suspend method 300 until such time as step 302 has been completed. Next, step 304, connects, confirms, indicates, or otherwise provides for a wireless charger (not shown) to be connected to a power source. Optionally, step 306 places, indicates, confirms or otherwise provides for transmitting portion of the wireless charger to be placed in pocket 202. If step 306 is not performed, the transmitting portion of the wireless charger may be placed proximate to wearable device 100 such as to energize the recharging antenna substantially located at recharging area icon 204. Step 308 then connects, indicates, confirms, or otherwise energizes wireless charger such as to charge a battery incorporated within wearable device 100. Optionally, steps 304 and 306 may be interchanged. Step 308 may cause one or more of charging indicator icon 104, charging indicator 106, battery status icon 108, battery status indicator 110 108 to be energized or otherwise indicate charging.

FIG. 4 depicts method 400 to operate a wearable device in accordance with embodiments of the present disclosure. In one embodiment, step 402 is powered up, such as by a user pressing power on-off button 102, wakeup button 124, etc. is received such as to energize wearable device 100. In one embodiment, at step 404, wearable device 100 entered a demonstration (demo) mode such as to cause one or more of the number of components of wearable device 100 to in parallel and/or serially execute instructions whereby components execute instructions to present operational options or features and/or receive inputs. Optionally, step 402 may comprise self-check or fault detection and/or indication. Next, in step 406 wearable device 100 may connect and communicate with an application (app) being executed by a secondary device operable to connect to wearable device 100, such as by BLE signals. In other embodiments, other wireless communications means (e.g., sound, infrared, other electromagnetic, etc.) and/or wired communication means (e.g., USB, CAT5/6/etc., Lightening, etc.) may be utilized. Steps 404 and 406 may be operated partially or entirely simultaneously or in a different order as that shown. Next, in step 408 a user provides inputs to the application, which in turn, operates wearable device 100.

In one example, wearable device 100 is turned on by toggling the on-off switch 101 to on, wearable device 100 enters demo mode for thirty seconds, during which wearable device operates the EL lighting 120 and POF lighting 116, display 118 and provide a message (e.g., “Flex”) on display 118. Optionally, a touch input in pressure sensor area 114 may cause display 118 to change to a different message (e.g., “touch”). In the demo mode, wearable device 100 may attempt to connect with an associated application such as via BLE signals. When connection is established, the demo mode terminates and wearable device 100 waits for instructions from the app and user inputs thereon.

In another embodiment, when pressing wakeup button 124, the device will enter or re-enter demo mode. When the demonstration is complete, wearable device 100 may be turned off via toggling on-off switch 102.

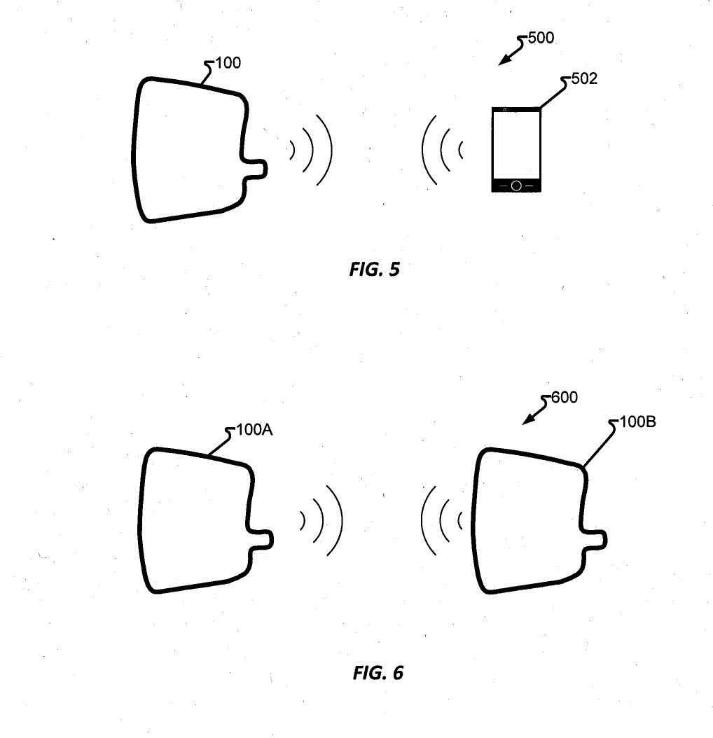

FIG. 5 depicts communication 500 utilizing wearable device 100 in accordance with embodiments of the present disclosure. In one embodiment, wearable device 100 communicates via wireless communication with other device 502. The communication may be via audible signals (e.g., tones, generated speech, etc.), light (e.g., visual and/or infrared link), and/or radio frequency (e.g., BlueTooth, etc.). Wearable device 100 and other device 502 may utilize each other for shared or segregated computing purposes. For example, other device 502 may comprise a cellular telephone or other networked communication device. Wearable device 100 may then, with benefit of the communication link with other device 502, communicate with nodes on the network. Other device 502 may be transmit-only device, such as Global Positioning Satellite/System (GPS) providing radio signals to the processor of wearable device 100 and, thereby providing location information for use by the user thereof or other components within or peripheral to wearable device 100.

FIG. 6 depicts communication 600 utilizing wearable devices 100A-B in accordance with embodiments of the present disclosure. In one embodiment, wearable device 100A communicates with wearable device 100B. For example, a user of wearable device 100A may send a message for display on wearable device 100B. Sensors or other inputs into wearable device 100A may be provided to wearable device 100B.

In one example, the user of wearable device 100A has been injured, such as may be detected by the user thereof interacting with pressure sensor 114 or by a decrease in body temperature determined by heater/temperature sensor 122. Accordingly, a processor of wearable device 100A may signal the processor of wearable device 100B to display a message (e.g., message on LED display 118, cause EL lighting 120 to illuminate or flash, etc.

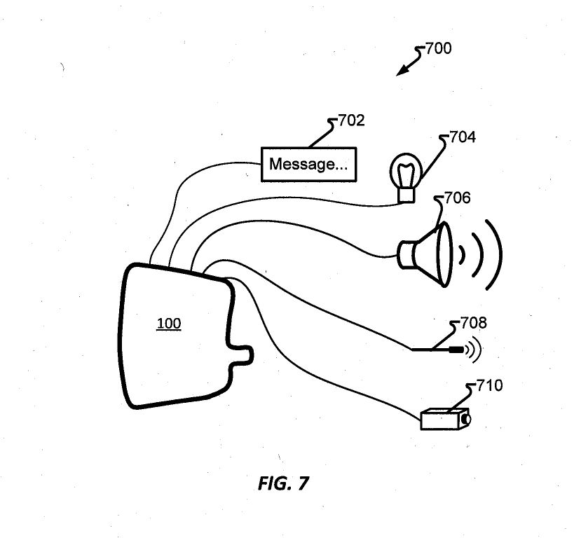

FIG. 7 depicts wearable device 700 utilizing a variety of peripheral devices. In one embodiment, wearable device 100 may incorporate or interface with, via wired and/or wireless connection, to a variety of peripheral devices. Peripheral devices may include input devices for sensing and/or detecting, such as microphone 708 or camera 710. Peripheral devices may include output devices for outputting signals, messages, etc. to a user and/or other components and may include, textual display 702, light 704, and/or speaker 706.

Camera 710 may, via the processor, provide cues to a user of wearable device 100. For example, a sight-impaired user may be provided with a tactile signal, such via an incorporated or peripheral vibration motor to indicate a direction to travel, a right (or wrong) turn, a hazard, a familiar face or location, etc.

One or more lights, such as light 704, EL lighting 120 and POF lighting 116 may be configured to convey a message and/or illumination for the user of wearable device 100 and/or other non-wearing user. For example, wearable device 100 may utilize one or more of the aforementioned light to display a pathway for the user to navigate at night or other low-light situations. In another example, lighting is provided by wearable device 100 to help motorists or other non-wearing users see wearable device 100 and the user thereof, such as when the user is walking close to a roadway at night.

Other input components that may be incorporated into, or in communication with, wearable device 100 may include an accelerometer, compass, or other directional/orientation sensing component. A user who has fallen (e.g., high level of acceleration and/or being in an orientation indicating the user is lying down, etc.) may cause such a component to indicate, via at least one output of the wearable device (e.g., EL lighting 120 and/or POF lighting 116) that the user is in distress and/or utilize a communication interface to, for example, send a text message or cellular telephone call to another party to provide an alert to the user's situation. Input area 114 may be configured to be placed on a joint of a user such that motion of the user provides an input to the processor of wearable device. For example, to indicate that a user should move their arm more (or less) often or to a greater (or lessor) amount as part of a therapy, rehabilitation, and/or training.

Embodiments herein are generally directed to a human user. However, in other embodiments, the user is non-human. For example, a service or rescue animal may be the user of wearable device 100 and be trained to respond to signals (e.g., visual cues, audible cues) from components such as EL lighting 120 and/or POF lighting 116. A non-human user may be able to provide tactile input (e.g., pecking, pawing, biting) pressure sensor area 114, which may be made to accommodate such an interaction without damage (e.g., protected by a thick rubber membrane, etc.). More specifically, many search and rescue animals are dogs that are capable of moving much faster over rough terrain than a human, however, a human handler usually keeps the dog on a lead or within close proximity, thereby limiting the speed of the dog. Continuous radio location of the dog may not be possible or desirable, however, by deploying wearable device 100, the dog may search without hinderance of a slower human and, communicate via an input to wearable device 100 causing a signal to be transmitted to indicate the subject has been found. In additional to triggering a radio signal, the dog may cause a light (e.g., EL lighting 120 and/or POF lighting 116) to illuminate or strobe in the visual and/or infrared spectrum to provide a visual cue to the human handler or other personnel as to the current location of the dog.

In yet another embodiment, wearable device 100 may be utilized by a mechanical or robotic system or component. For example, a robot with wearable device 100 may touch a human and thereby cause pressure sensor area 114 to be energized and respond according (e.g., stop, reverse, engage another component, etc.).

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

Any of the steps, functions, and operations discussed herein can be performed continuously and automatically.

The exemplary systems and methods of this disclosure have been described in relation to a smart wearable component. However, to avoid unnecessarily obscuring the present disclosure, the preceding description omits a number of known structures and devices. This omission is not to be construed as a limitation of the scopes of the claims. Specific details are set forth to provide an understanding of the present disclosure. It should however be appreciated that the present disclosure may be practiced in a variety of ways beyond the specific detail set forth herein.

Furthermore, while the exemplary aspects, embodiments, and/or configurations illustrated herein show the various components of the system collocated, certain components of the system can be located remotely, at distant portions of a distributed network, such as a LAN and/or the Internet, or within a dedicated system. Thus, it should be appreciated, that the components of the system can be combined in to one or more devices, such as a wearable, or collocated on a particular node of a distributed network, such as an analog and/or digital telecommunications network, a packet-switch network, or a circuit-switched network. It will be appreciated from the preceding description, and for reasons of computational efficiency, that the components of the system can be arranged at any location within a distributed network of components without affecting the operation of the system. For example, the various components can be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

Also, while the processes discussed in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, subcombinations and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A wearable device comprising:
an article of clothing integrating the wearable device for use by a user while wearing the article of clothing;
a flexible battery providing electrical power to the wearable device;
a flexible output providing a data output;
a flexible input receiving a data input; and
a processor to provide the data output in response to the data input and powered by the flexible battery.

2. The wearable device of claim 1, wherein a portion of the article of clothing conceals the wearable device.

3. The wearable device of claim 3, further comprising at least one of an inductive power receiver or a magnetic power receiver configured to, when energized by the inductive power source, supply electrical power to the flexible battery for charging the flexible battery.

4. The wearable device of claim 3, further comprising a pocket to receive at least one of an inductive power source or a magnetic power source and restrict the motion of the at least one of inductive power source or magnetic power source while within the pocket and thereby maintain a proximity between the at least one inductive power source or magnetic power source sufficient to enable inductive and/or magnetic coupling between the at least one of the inductive power source and the inductive power receiver or the magnetic power source and the magnetic power receiver.

5. The wearable device of claim 1, further comprising a data connectivity port configured to, when connected to an external device, facilitate data communication between the processor and the external device.

6. The wearable device of claim 1, wherein the article of clothing is affixed to the wearable device with user-operable attachments.

7. The wearable device of claim 1, further comprising:
a temperature sensor; and
wherein the temperature sensor is connected, for data communication, with the processor.

8. The wearable device of claim 1, wherein the flexible output and the flexible input are configured to communicate with a secondary device executing an application for operating the wearable device.

9. The wearable device of claim 1, wherein the processor causes the wearable device to operate in a demonstration mode.

10. A wearable device comprising:
a flexible battery providing electrical power to the wearable device;
a flexible output providing a data output;
a flexible input receiving a data input; and
a processor to provide the data output in response to the data input and powered by the flexible battery.

11. The wearable device of claim 10, wherein the flexible input comprises a pressure sensor.

12. The wearable device of claim 10, further comprising:
a flexible printed circuit board to accommodate one or more electronic components disposed thereon.

13. The wearable device of claim 10, further comprising, an antenna to perform at least one of receiving or transmitting radio frequency signals.

14. The wearable device of claim 13, wherein the antenna is operable to perform the at least one of receiving or transmitting radio frequency signals comprising low power Bluetooth signals.

15. The wearable device of claim 10, wherein the flexible output comprises a display.

16. The wearable device of claim 10, wherein the flexible output comprises an electroluminescent tape (EL) lighting.

17. The wearable device of claim 10, wherein the flexible input comprises a temperature sensor.

18. The wearable device of claim 10, wherein the flexible output and the flexible input are configured to communicate with a secondary device executing an application for operating the wearable device.

19. The wearable device of claim 10, wherein the processor causes the wearable device to operate in a demonstration mode.

20. A method for operating a wearable device, comprising:
energizing the wearable device;
operating, by a processor of the wearable device, the wearable device in a demonstration mode;
during at least a portion of the demonstration mode, connecting via wireless component to a second device executing an application for the operation of the wearable device; and
executing an operation corresponding to a user input received by the application executing on the secondary device and transmitted via wireless connection to the wearable device.
